(19) United States
(12) Patent Application Publication
(10) Pub. No.: US 2004/0077515 A1
(43) Pub. Date: Apr. 22, 2004

(54) Bis-triazinylaminobenzoxazole derivatives

(76) Inventors: Georges Metzger, Moernach (FR); Serge Hauger, Ranspach-le-Bas (FR); Fabienne Cuesta, Roppentzwiller (FR)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/468,278

(22) PCT Filed: Feb. 12, 2002
(86) PCT No.: PCT/EP02/01442

(30) Foreign Application Priority Data
Feb. 21, 2001 (EP) 01810181.6

(51) Int. Cl. C11D 7/42; C07D 413/14

(52) U.S. Cl. 510/394; 544/198; 544/113

(57) ABSTRACT

The present invention relates to novel compounds represented by the formula (1): wherein R₁ and R₂, independently, represent —NH₂, NH(C₁-C₄alkyl), —N(C₁-C₄alkyl)₂, —NH(C₂-C₄hydroxyalkyl), —N(C₂-C₄hydroxyalkyl)₂, —N(C₁-C₄alkyl)(C₂-C₄hydroxyalkyl), —NH(C₁-C₄alkoxy-C₂-C₄hydroxyalkyl), —N(C₁-C₄alkoxy-C₂-C₄hydroxyalkyl)₂, —N(C₁-C₄alkoxy-C₂-C₄hydroxyalkyl)(C₁-C₄alkyl), —N(C₁-C₄alkkoxy-C₂-C₄hydroxyalkyl)(C₂-C₄hydroxyalkyl), NH(C₁-C₄alkoxy-C₁-C₄-alkoxy-C₁-C₄alkyl), —N(C₁-C₄alkoxy-C₁-C₄alkkoxy-C₁-C₄-alkyl)₂, —N(C₁-C₄alkoxy-C₁-C₄alkoxy-C₁-C₄-alkyl)(C₁-C₄alkyl), —N(C₁-C₄alkoxy-C₁-C₄alkoxy-C₂-C₄-alkyl)(C₂-C₄hydroxyalkyl) —NH(C₅-C₇cycloalkyl), —N(C₅-C₇cycloakyl)₂, —NH(C₆-C₁₀aryl), NH(C₇-C₁₃-aralkyl) or a morpholino, piperidino or pyrrolidino residue; halogen or —OC₁-C₄alkyl, which is unsubstituted or substituted by hydroxy or —C₁-C₄alkoxy, a process for their preparation and their use as fluorescent whitening agents (FWA's) for natural or synthetic materials.

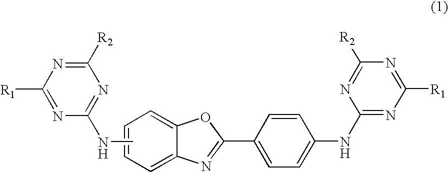

The present invention relates to novel bis-triazinylaminobenzoxazole derivatives, a process for their preparation and their use as fluorescent whitening agents (FWA's) for natural or synthetic materials.

The novel compounds are represented by the formula

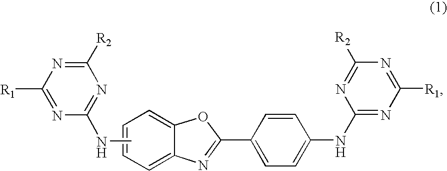

wherein

R₁ and R₂, independently, represent —NH₂, —NH(C₁-C₄alkyl), —N(C₁-C₄alkyl)₂, —NH(C₂-C₄hydroxyalkyl), —N(C₂-C₄hydroxyalkyl)₂, —N(C₁-C₄alkyl)(C₂-C₄hydroxyalkyl), —NH(C₁-C₄alkoxy-C₂-C₄ hydroxyalkyl), —N(C₁-C₄alkoxy-C₂-C₄ hydroxyalkyl)₂, —N(C₁-C₄alkoxy-C₂-C₄ hydroxyalkyl) (C₁-C₄alkyl), —N(C₁-C₄alkoxy-C₂-C₄ hydroxyalkyl)(C₂-C₄hydroxyalkyl), —NH(C₁-C₄alkoxy-C₁-C₄alkoxy-C₁-C₄-alkyl), —N(C₁-C₄alkoxy-C₁-C₄alkoxy-C₁-C₄-alkyl)₂, —N(C₁-C₄alkoxy-C₁-C₄alkoxy-C₁-C₄-alkyl)(C₁-C₄alkyl), —N(C₁-C₄alkoxy-C₁-C₄alkoxy-C₂-C₄-alkyl)(C₂-C₄ hydroxyalkyl) —NH(C₅-C₇cycloalkyl), —N(C₅-C₇cycloalkyl)₂, —NH(C₆-C₁₀aryl), NH(C₇-C₁₃aralkyl) or a morpholino, piperidino or pyrrolidino residue; halogen or —OC₁-C₄alkyl, which is unsubstituted or substituted by hydroxy or —C₁-C₄alkoxy.

Preferred compounds of the formula (1) are represented by the formula

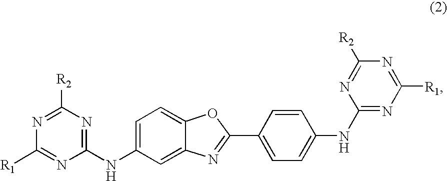

in addition to those of the formula

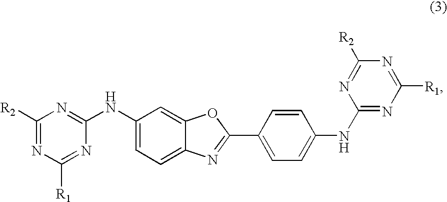

in which

R₁ and R₂ are as defined previously.

Within the scope of the compounds of formulae (1) to (3), such compounds in which R₁ represents —NH₂, —NH(C₁-C₄alkyl), —N(C₁-C₄alkyl)₂, —NH(C₂-C₄hydroxyalkyl), —N(C₂-C₄hydroxyalkyl)₂, —N(C₁-C₄alkyl)(C₂-C₄hydroxyalkyl), —NH(C₁-C₄alkoxy-C₂-C₄ hydroxyalkyl), —N(C₁-C₄alkoxy-C₂-C₄ hydroxyalkyl)₂, —N(C₁-C₄alkoxy-C₂-C₄ hydroxyalkyl) (C₁-C₄alkyl), —N(C₁-C₄alkoxy-C₂-C₄ hydroxyalkyl)(C₂-C₄hydroxyalkyl), —NH(C₁-C₄alkoxy-C₁-C₄alkoxy-C₁-C₄-alkyl), —N(C₁-C₄alkoxy-C₁-C₄alkoxy-C₁-C₄-alkyl)₂, —N(C₁-C₄alkoxy-C₁-C₄alkoxy-C₁-C₄-alkyl)(C₁-C₄alkyl), —N(C₁-C₄alkoxy-C₁-C₄alkoxy-C₂-C₄-alkyl)(C₂-C₄ hydroxyalkyl) —NH(C₅-C₇cycloalkyl), —N(C₅-C₇cycloalkyl)₂, NH(C₇-C₁₃aralkyl) or a morpholino, piperidino or pyrrolidino residue; halogen or —OC₁-C₄alkyl, which is unsubstituted or substituted by hydroxy or —C₁-C₄alkoxy and R₂ represents —NH(C₆-C₁₀aryl) are of particular interest and, more especially, compounds in which R₂ represents an anilino residue which is unsubstituted or substituted by one or two —SO₃M groups, M representing H, Na, Li, K, Ca, Mg, ammonium, or ammonium that is mono-, di-, tri- or tetrasubstituted by C₁-C₄alkyl, C₂-C₄hydroxyalkyl or a mixture thereof.

Most especially preferred compounds are those in which R₁ represents —NH₂, —NH(C₁-C₄alkyl), —N(C₁-C₄alkyl)₂, —NH(C₂-C₄hydroxyalkyl), —N(C₂-C₄hydroxyalkyl)₂, NH(C₇-C₁₃aralkyl) or a morpholino, piperidino or pyrrolidino residue; chlorine or —OC₁-C₄alkyl, which is unsubstituted or substituted by hydroxy or —C₁-C₄alkoxy, whereby, in particular, R₁ represents —NH(C₁-C₄alkyl), —N(C₁-C₄alkyl)₂, a morpholino residue; chlorine or —OC₁-C₄alkyl, which is unsubstituted or substituted by hydroxy, ethoxy or methoxy and R₂ represents an anilino residue which is unsubstituted or substituted by one —SO₃M group, M representing H, Na, or K.

Within the scope of the definitions of the substituents R₁ and/or R₂, C₁-C₄alkyl groups may be branched or unbranched such as methyl, ethyl, n-propyl, isopropyl, n-butyl, sec-butyl, isobutyl or t-butyl.

Similarly, C₁-C₄alkoxy groups may be branched or unbranched such as methoxy, ethoxy, n-propoxy, isopropoxy, n-butoxy, sec-butoxy, isobutoxy or t-butoxy, whilst there may be defined as C₂-C₄hydroxyalkyl, hydroxyethyl, hydroxypropyl or hydroxybutyl.

As C₅-C₇cycloalkyl, there are defined cyclopentyl, cyclohexyl or cycloheptyl, whilst C₆-C₁₀aryl may be phenyl, which is unsubstituted or substituted by one or more C₁-C₄alkyl, C₁-C₄alkoxy or SO₃M groups or, alternatively, naphthalene, which may be substituted by one or more SO₃M groups and C₇-C₁₃aralkyl is, for example, substituted or unsubstituted benzyl, phenethyl, naphthylmethyl or naphthylethyl.

Halogen, in compounds of formulae (1) to (3), is bromine, fluorine or, especially, chlorine.

A further aspect of the present invention is a process for the preparation of the compound of formula (1) by reacting, cyanuric chloride, successively, in any desired sequence, with a diaminobenzoxazole of the formula

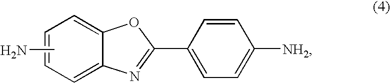

a compound capable of introducing a group R₁ and a compound capable of introducing a group R₂, in which R₁ and R₂ are as previously defined.

Compounds of formula (4) are known or may be prepared by known methods.

In dissolved or finely divided states, the compounds of formula (1) display a more or less pronounced fluorescence. They are therefore used, according to the invention, for optically brightening synthetic or natural organic materials.

Examples of such materials which may be mentioned, without the review given below being intended to express any limitation thereto, are textile fibres from the following groups of organic materials, insofar as optical brightening thereof enters into consideration:

(a) Polyamides which are obtainable as polymerisation products by ring opening, for example those of the polycaprolactam type,

(b) polyamides which are obtainable as polycondensation products based on bifunctional or polyfunctional compounds capable of undergoing a condensation reaction, such as hexamethylenediamine adipate and

(c) natural textile organic materials of animal or vegetable origin, for example based on cellulose or proteins, such as cotton or wool, linen or silk.

The organic materials to be optically brightened can be in diverse stages of processing and are preferably finished textile products. They can, for example be in the form of hank goods, textile filaments, yarns, twisted yams, nonwovens, felts, textile fabrics, textile composites or knitted fabrics.

The brighteners defined above are of particular importance for the treatment of textile fabrics. The treatment of textile substrates is advantageously carried out in an aqueous medium in which the particular optical brighteners are present in a finely divided form (suspensions, so-called microdispersions and in some cases solutions). Dispersing agents, stabilisers, wetting agents and further auxiliaries can optionally be added during the treatment.

The treatment is usually carried out at temperatures of from about 20° to 140° C., for example at the boiling point of the bath, or in the region thereof (about 90° C.). For the finishing, according to the invention, of textile substrates it is also possible to use solutions or emulsions in organic solvents, as are used in dyeing practice in so-called solvent dyeing (pad-thermofix method and the exhaustion dyeing process in dyeing machines).

The optical brighteners which can be used according to the present invention can also be employed, for example, in the following use forms:

(a) In mixtures with so-called “carriers”, wetting agents, softeners, swelling agents, antioxidants, light stabilisers, heat stabilisers and chemical bleaching agents (chlorite bleach and bleaching bath additives).

(b) In mixtures with crosslinking agents and finishing agents (for example starch or synthetic finishing agents) and also in combination with very diverse textile finishing processes, especially synthetic resin finishes (for example crease resistant finishes such as “wash-and-wear”, “permanent press” and “no-iron”), and also flame resistant finishes, soft handle finishes, anti-soiling finshes or anti-static finishes or antimicrobial finishes.

(c) As additives to various soaps and washing agents.

(d) In combination with other substances having an optical brightening action.

If the brightening process is combined with textile treatment or finishing methods, the combined treatment can in many cases advantageously be effected with the aid of corresponding stable formulations which contain the compounds having an optical brightening action in a concentration such that the desired brightening effect is obtained.

In certain cases, the full effect of the brightener is achieved by an after-treatment. This can be, for example, a chemical treatment (for example acid treatment), a thermal treatment (for example heat) or a combined chemical/heat treatment.

The amount of the optical brighteners to be used according to the invention, relative to the material to be optically brightened, can vary within wide limits. A distinct and durable effect can already be achieved with vary small amounts and in certain cases, for example, with amounts of 0.03% by weight. However amounts of up to about 0.5% by weight can also be used. For most cases of interest in practice, amounts of between 0.05 and 0.5% by weight relative to the material to be brightened, are preferably of interest.

The optical brighteners are also especially suitable as additives for washing baths or to industrial and household washing agents and they can be added in various ways. They are appropriately added to washing baths in the form of their solutions in water or organic solvents or also in a state of fine division as aqueous dispersions or slurries. They, or their components, are advantageously added to household or industrial washing agents at any phase of the manufacturing process of the washing agent, for example to the so-called “slurry” prior to spray-drying of the washing powder or during the preparation of liquid washing agent combinations. The compounds can be added both in the form of a solution or dispersion in water or other solvents and also without auxiliaries in the form of a dry brightener powder. However, they can also be sprayed, in the dissolved or pre-dispersed form, onto the finished washing agent.

Washing agents which can be used are the known mixtures of detergent substances, such as, for example, soap in the form of chips and powders, synthetic products, soluble salts of sulphonic acid half-esters of higher fatty alcohols, arylsulphonic acids, which are substituted by higher alkyl and/or polysubstituted by alkyl, carboxylic acid esters with alcohols of medium to higher molecular weight, fatty acid acylaminoalkyl- or aminoaryl-glycerol-sulphonates, phosphoric acid esters of fatty alcohols and the like. So-called “builders” which can be used are, for example, alkali metal polyphosphates and alkali metal polymeta-phosphates, alkali metal pyrophosphates, alkali metal salts of carboxyethylcellulose and other “soil redeposition inhibitors”, and also alkali metal silicates, alkali metal carbonates, alkali metal borates, alkali metal perborates, nitrilotriacetic acid, ethylenediamine-tetraacetic acid and foam stabilisers, such as alkanolamides of higher fatty acids. Furthermore, the washing agents can contain, for example: antistatic agents, superfatting skin protection agents, such as lanolin, enzymes, antimicrobial agents, perfumes and dyestuffs.

The brighteners have the particular advantage that they are also effective in the presence of active chlorine donors, such as, for example, hypochlorite and can be used without substantial loss of the effects in washing baths with non-ionic washing agents, for example alkylphenol polyglycol ethers. Also in the presence of perborate or peracids and activators, for example tetraacetylglycoluril or ethylenediamine-tetraacetic acid ar the new brighteners very stable both in pulverulent washing agent and in washing baths.

The brighteners according to the invention are added in amounts of 0.005 to 2% or more and preferably of 0.03 to 0.5%, relative to the weight of the liquid or pulverulent ready-to-use washing agent. When they are used to wash textiles made of cellulose fibres, polyamide fibres, cellulose fibres with a high grade finish, wool and the like, wash liquors which contain the indicated amounts of the optical brighteners according to the invention impart a brilliant appearance in daylight.

The washing treatment is carried out, for example, as follows:

The indicated textiles are treated for 1 to 30 minutes at 5° to 100° C . and preferably at 25° to 100° C. in a wash bath which contains 1 to 10 g/kg of a composite washing agent containing builders and 0.05 to 1% relative to the weight of the washing agent, of the brighteners claimed. The liquor ratio can be 1:3 to 1:50. After washing, the textiles are rinsed and dried in the customary manner. The wash bath can contain, as a bleach additive, 0.2 g/l of active chlorine (for example in the form of hypochlorite) or 0.1 to 2 g/l of sodium perborate.

The brighteners according to the invention can also be applied from a rinsing bath with a “carrier”. For this purpose the brightener is incorporated in a soft rinsing agent or in another rinsing agent, which contains, as the “carrier”, for example, polyvinyl alcohol, starch, copolymers on an acrylic basis or formaldehyde/urea or ethylene-urea or propylene-urea derivatives, in amounts of 0.005 to 5% or more and preferably of 0.2 to 2%, relative to the rinsing agent. When used in amounts of 1 to 100 ml, and, preferably of 2 to 25 ml, per litre of rinsing bath, rinsing agents of this type, which contain the brighteners according to the invention, impart brilliant brightening effects to very diverse types of treated textiles.

A further application of the compounds of the invention is for the brightening of paper, either in the pulp mass during paper manufacture or in the size-press, which has been described in British Patent Specification 1,247,934, in the film press or, preferably, in coating compositions. When brighteners of the present invention are employed in such formulations papers brightened with them exhibit a very high degree of whiteness.

The compounds obtained by the process of the present invention are particularly advantageous in that they exhibit not only extremely high whitening ability, but, in addition, in many cases highly desirable water solubilities and also possess excellent white aspects in the solid state.

The following Examples serve to illustrate the invention; parts and percentages are by weight, unless otherwise stated.

### EXAMPLE 1

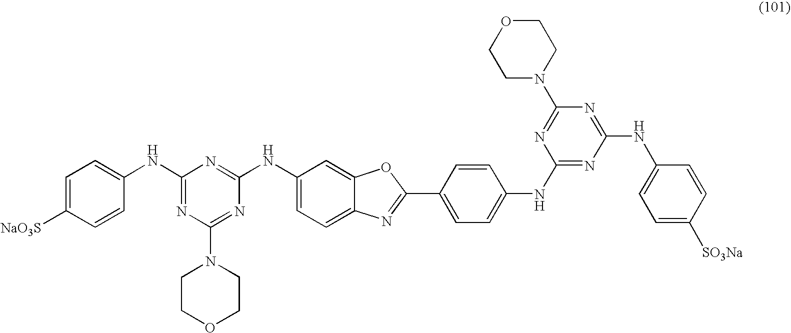

5.5 g of cyanuric chloride are dissolved in 39 ml of methyl ethyl ketone and the solution onto 27 g of ice. To the resulting suspension, a solution of 5.3 g of sulphanilic acid dissolved in 14.85 g of 2N sodium hydroxide solution and 40 g of water is added at pH 3.5, followed by 14.85 ml of a 1M sodium carbonate solution over 15 minutes at 0° C. To the resulting mixture, a solution of 13.6 mMoles of 2-(4-aminophenyl)-6-aminobenzoxazole, prepared as described in Helv. Chim. Acta, 63, 413-419 (1980), in 27 ml of water is added and the pH adjusted to 9.0 by the addition of a 1M solution of sodium carbonate. The temperature is then raised to 40° C. over 3 hours, the pH being held constant. After this time, 4.6 g of morpholine are added and the temperature raised to 99° C., the methyl ethyl ketone being distilled off and the pH being maintained at 9.5.

After cooling to 40° C., 600 ml of ethanol are stirred into the reaction mixture and the precipitated product filtered off and dried. There are obtained 8.9 g of very pure product of formula (101) as a white powder, characterized by the following elemental analysis:

C₃₉H₃₅N₁₃Na₂O₉S₂.6.5H₂O. 0.2 NaCl requires: C, 43.83%; H, 4.53%; Cl, 0.66%; N, 17.04%; Na, 4.73% and S, 6.00%; found: C, 43.94%; H, 4.35%; Cl, 0.66%; N, 17.05%; Na, 4.64% and S, 6.01%. and by the following UV spectral data recorded in dimethylformamide/water 1:1 Absorption λₘₐₓ 355 nm, ε 62,800; Fluorescence λₘₐₓ 420 nm, Quantum Yield φ 0.83.

### EXAMPLE 2

By following the procedure described in Example 1, but performing the work-up procedure prior to addition of the morpholine, the following compound of formula (102) is obtained:

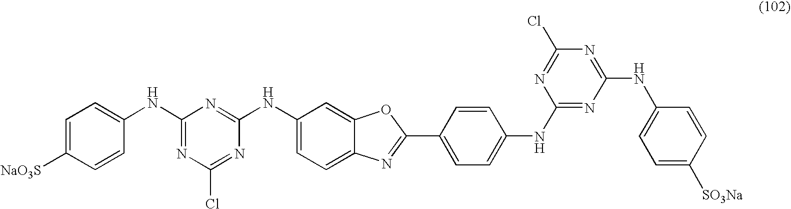

### EXAMPLES 3-6

By following the procedure described in Example 1, but replacing the morpholine by equivalent quantities of methylamine, ethylene glycol, methoxy ethanol and 3-dimethylaminopropylamine, respectively, the compounds of formula (5) described In the following Table 1 are obtained:

| | |
|---|---|
| | (5) |
| 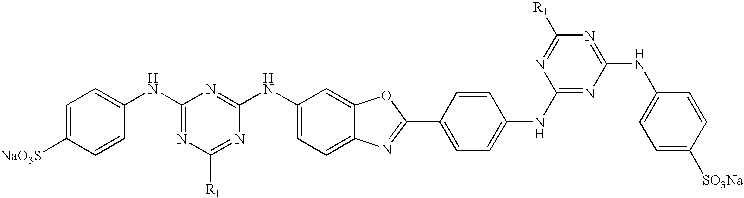 | |

| Example Nr. | R₁ |
|---|---|
| (103) | —NHCH₃ |
| (104) | —OCH₂CH₂OH |
| (105) | —OCH₂CH₂OCH₃ |
| (106) | —NHCH₂CH₂CH(NCH₂CH₃)₂ |

### EXAMPLE 7-10

By following the procedure described in Example 1, but replacing the 2-(4-aminophenyl)-6-aminobenzoxazole by 2-(4-aminophenyl)-5-aminobenzoxazole, obtained as described in Helv. Chim. Acta, 63, 413-419 (1980), and either proceeding as described in Example 2 (for compound 109) or employing, appropriately, morpholine, methylamine or ethylene glycol in the final reaction step, the compounds of formula (6) described in the following Table 2 are obtained:

TABLE 2

| | |
|---|---|
| | (6) |
| 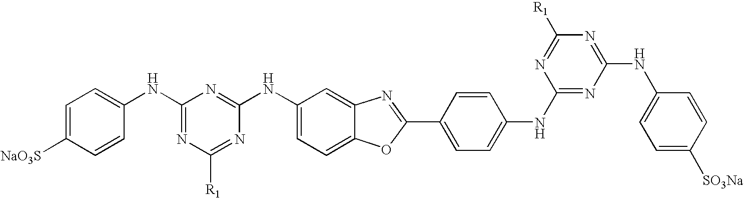 | |

| Example Nr. | R₁ |
|---|---|
| (107) | morpholino |
| (108) | —NHCH₃ |
| (109) | —Cl |
| (110) | —OCH₂CH₂OH |

1. A compound of the formula 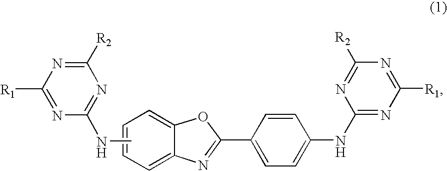
wherein
R₁ and R₂, independently, represent —NH₂, —NH(C₁-C₄alkyl), —N(C₁-C₄alkyl)₂, —NH(C₂-C₄hydroxyalkyl), —N(C₂-C₄hydroxyalkyl)₂, —N(C₁-C₄alkyl)(C₂-C₄hydroxyalkyl), —NH(C₁-C₄alkoxy-C₂-C₄ hydroxyalkyl), —N(C₁-C₄alkoxy-C₂-C₄ hydroxyalkyl)₂, —N(C₁-C₄alkoxy-C₂-C₄ hydroxyalkyl) (C₁-C₄alkyl), —N(C₁-C₄alkoxy-C₂-C₄ hydroxyalkyl)(C₂-C₄hydroxyalkyl), —NH(C₁-C₄alkoxy-C₁-C₄alkoxy-C₁-C₄alkyl), —N(C₁-C₄alkoxy-C₁-C₄alkoxy-C₁-C₄-alkyl)₂, —N(C₁-C₄alkoxy-C₁-C₄alkoxy-C₁-C₄-alkyl)(C₁-C₄alkyl), —N(C₁-C₄alkoxy-C₁-C₄alkoxy-C₂-C₄-alkyl)(C₂-C₄ hydroxyalkyl) —NH(C₅-C₇cycloalkyl), —N(C₅-C₇cycloalkyl)₂, —NH(C₆-C₁₀aryl), NH(C₇-C₁₃aralkyl) or a morpholino, piperidino or pyrrolidino residue; halogen or —OC₁-C₄alkyl, which is unsubstituted or substituted by hydroxy or —C₁-C₄alkoxy.

2. A compound of the formula 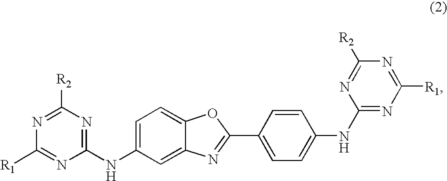
in which
R₁ and R₂ are as defined in claim 1.

3. A compound of the formula 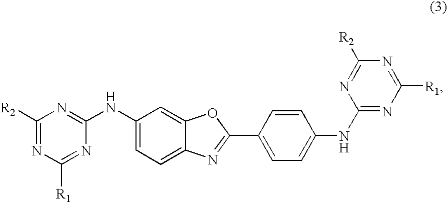
in which
R₁ and R₂ are as defined in claim 1.

4. A compound according to any one of claims 1-3, wherein
R₁ represents —NH₂, —NH(C₁-C₄alkyl), —N(C₁-C₄alkyl)₂, —NH(C₂-C₄hydroxyalkyl), —N(C₂-C₄hydroxyalkyl)₂, —N(C₁-C₄alkyl)(C₂-C₄hydroxyalkyl), —NH(C₁-C₄alkoxy-C₂-C₄ hydroxyalkyl), —N(C₁-C₄alkoxy-C₂-C₄ hydroxyalkyl)₂, —N(C₁-C₄alkoxy-C₂-C₄ hydroxyalkyl) (C₁-C₄alkyl), —N(C₁-C₄alkoxy-C₂-C₄ hydroxyalkyl)(C₂-C₄hydroxyalkyl), —NH(C₁-C₄alkoxy-C₁-C₄alkoxy-C₁-C₄-alkyl), —N(C₁-C₄alkoxy-C₁-C₄alkoxy-C₁-C₄-alkyl)₂, —N(C₁-C₄alkoxy-C₁-C₄alkoxy-C₁-C₄-alkyl)(C₁-C₄alkyl), —N(C₁-C₄alkoxy-C₁-C₄alkoxy-C₂-C₄-alkyl)(C₂-C₄ hydroxyalkyl) —NH(C₅-C₇cycloalkyl), —N(C₅-C₇cycloalkyl)₂, NH(C₇-C₁₃aralkyl) or a morpholino, piperidino or pyrrolidino residue;
halogen or —OC₁-C₄alkyl, which is unsubstituted or substituted by hydroxy or —C₁-C₄alkoxy and
R₂ represents —NH(C₈-C₁₀aryl).

5. A compound according to claim 4, in which
R₂ represents an anilino residue which is unsubstituted or substituted by one or two —SO₃M groups,
M representing H, Na, Li, K, Ca, Mg, ammonium, or ammonium that is mono-, di-, tri- or tetrasubstituted by C₁-C₄alkyl, C₂-C₄hydroxyalkyl or a mixture thereof.

6. A compound according to claim 5, in which
R₁ represents —NH₂, —NH(C₁-C₄alkyl), —N(C₁-C₄alkyl)₂, —NH(C₂-C₄hydroxyalkyl), —N(C₂-C₄hydroxyalkyl)₂, NH(C₇-C₁₃aralkyl) or a morpholino, piperidino or pyrrolidino residue; chlorine or —OC₁-C₄alkyl, which is unsubstituted or substituted by hydroxy or —C₁-C₄alkoxy.

7. A compound according to claim 6, in which
R₁ represents —NH(C₁-C₄alkyl), —N(C₁-C₄alkyl)₂, a morpholino residue; chlorine or —OC₁-C₄alkyl, which is unsubstituted or substituted by hydroxy, ethoxy or methoxy and R₂ represents an anilino residue which is unsubstituted or substituted by one —SO₃M group, M representing H, Na, or K.

8. A process for the preparation of the compound of formula (1) by reacting, cyanuric chloride, successively, in any desired sequence, with a diaminobenzoxazole of the formula 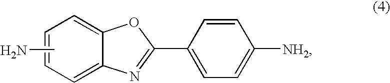
a compound capable of introducing a group R₁ and a compound capable of introducing a group R₂, in which R₁ and R₂ are as defined in claim 1.

9. Use of the compounds of formula (1) according to claim 1 as optical brightening agents for synthetic or natural organic materials.

10. Use of the compounds of formula (1) according to claim 1 as optical brightening agents for paper in pulp, size-press, film press or coating applications.

11. Use of the compounds of formula (1) according to claim 1 as optical brightening agents for textile materials.

12. Use of the compounds of formula (1) according to claim 1 as optical brightening agents in detergent compositions.
